(19) United States
(12) Patent Application Publication
(10) Pub. No.: US 2006/0206076 A1
(43) Pub. Date: Sep. 14, 2006

(54) Sanitary pad with large absorbing area

(76) Inventor: Wen-Lin Chiang, Taichung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/076,930

(22) Filed: Mar. 11, 2005

(51) Int. Cl. A61F 13/15

(52) U.S. Cl. 604/385.01

(57) ABSTRACT

A sanitary pad includes an elongate body having a front end, a rear end and two divergent and smooth sides connected between the front end and the rear end. The first side of the sanitary pad has an absorbing area, wherein the area of the front end is narrower than the area of the rear end which is used to cover the user's buttocks. An elongate reinforcement absorbing area is located at the first side and close to the front end. An adherent layer is applied to a second side of the body so as to attach the sanitary pad to the user's panties.

### FIELD OF THE INVENTION

The present invention relates to a sanitary pad with a large absorbing area at the rear end so as to cover a larger area of the user's buttocks.

### BACKGROUND OF THE INVENTION

A conventional sanitary pad is disclosed in FIG. 6 and generally includes an elongate absorbing area 20 and two wings 21 located on two sides of the absorbing area 20. The absorbing area 20 is composed of absorbent for absorbing liquid during the menses and the wings 21 includes adherent surface so as to attach the sanitary pad to the panties. However, the absorbing area 20 is not long and wide enough at the rear end thereof so that it cannot meet needs for night use and/or when sleeping. Besides, the wings 21 are located at a middle portion of the sanitary pad and include limited area so that the sanitary pad might move when in activity.

The present invention intends to provide an improved sanitary pad which includes a large absorbing area at the rear end of the pad and an adherent layer is applied on the opposite side of the absorbing area.

### SUMMARY OF THE INVENTION

The present invention relates to a sanitary pad that covers a large area of the user's buttocks and comprises an elongate body having a front end, a rear end and two divergent and smooth sides connected between the front end and the rear end. An absorbing area is defined in a first side of the body and an area of the front end is narrower than an area of the rear end. An elongate reinforcement absorbing area is located at the first side and close to the front end. An adherent layer is applied to a second side of the body and located opposite to the first side.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view of the sanitary pad of the present invention;

FIG. 2 is a top view of the sanitary pad of the present invention;

FIG. 3 is a side view of the sanitary pad of the present invention;

FIG. 4 shows that the sanitary pad of the present invention is attached to a panty;

FIG. 5 shows the area that the sanitary pad of the present invention covers, and

FIG. 6 shows a conventional sanitary pad.

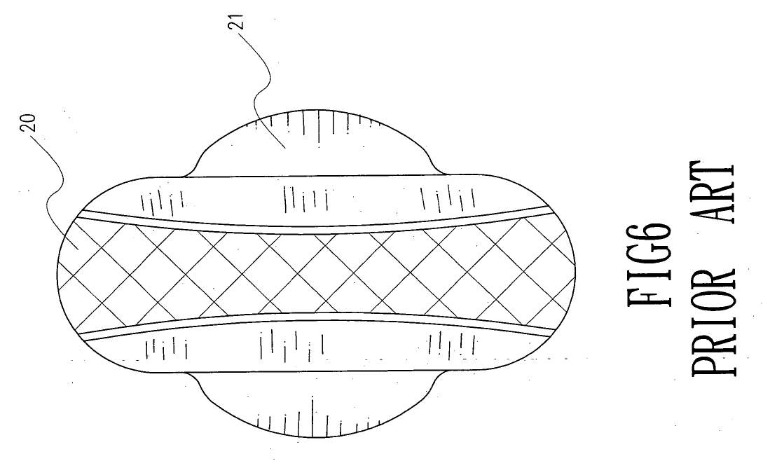

### DETAILED DESCRIPTION OF THE PREFERRED EMBODIMENT

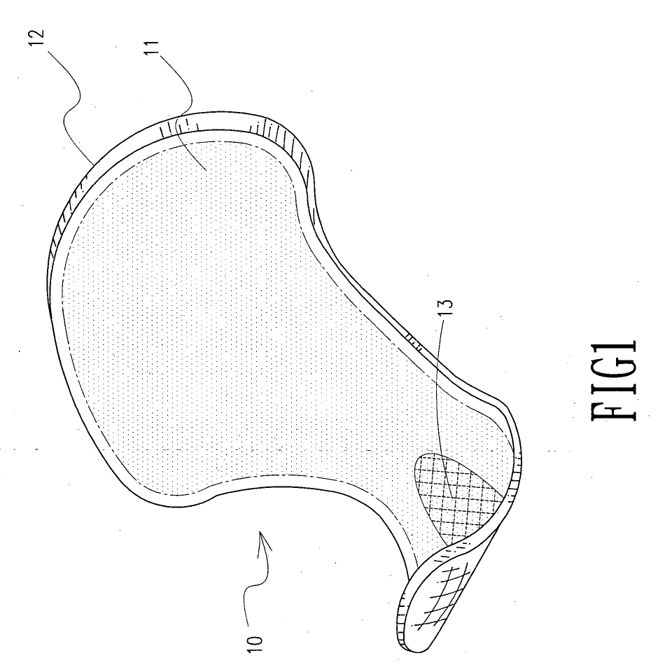

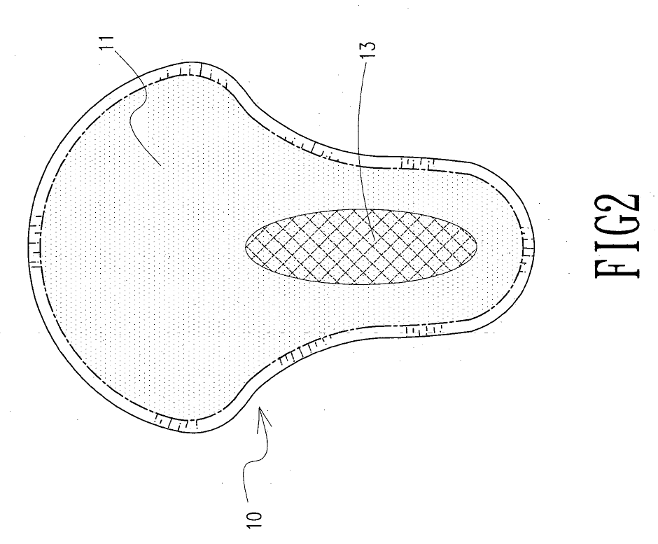

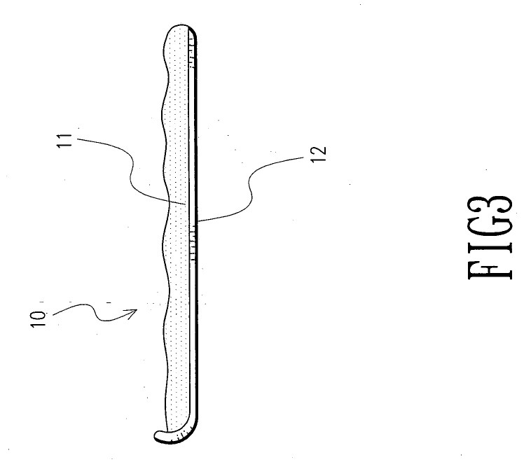

Referring to FIGS. 1 to 3, the sanitary pad 10 of the present invention comprises an elongate body having a front end, a rear end and two divergent and smooth sides are connected between the front end and the rear end. An absorbing area 11 is defined in a first side of the body and an area of the front end is narrower than an area of the rear end of the sanitary pad 10. An elongate reinforcement absorbing area 13 is located at the first side and close to the front end. An adherent layer 12 is applied to a second side of the body and located opposite to the first side.

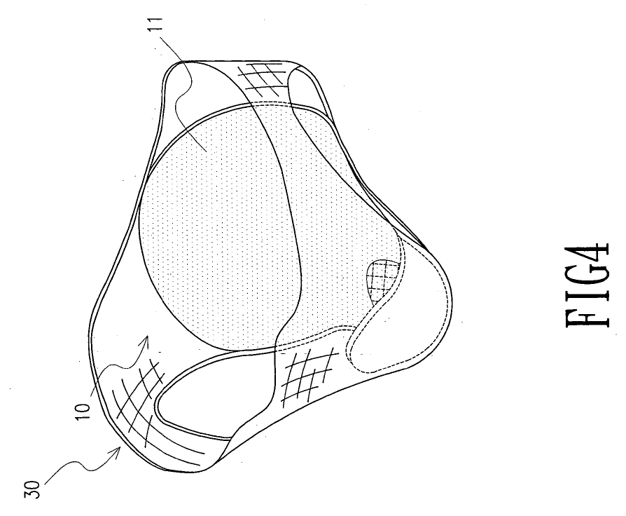

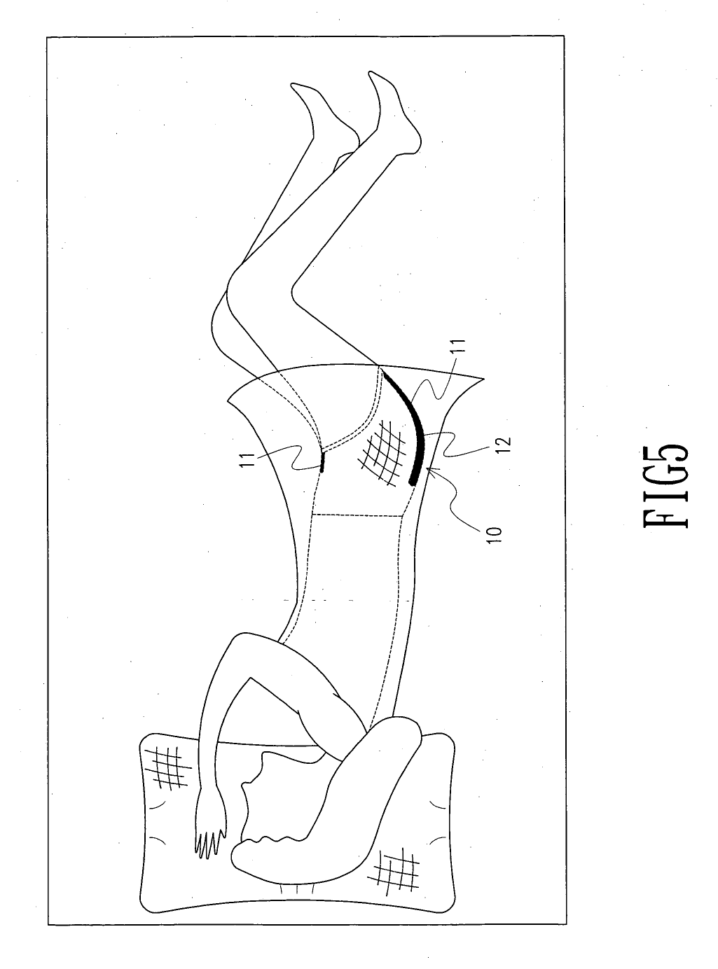

As shown in FIGS. 4 and 5, the sanitary pad 10 is conveniently attached on the panty by the adherent layer 12 which attaches the whole sanitary pad 10 to the panty so that the sanitary pad 10 does not move. The rear end of the sanitary pad 10 covers the user's buttocks so that it effectively absorbs liquid when the user is sleeping. The sanitary pad 10 provides solutions of the inherent shortcomings of conventional sanitary pad and makes the users not to worry about displacement and leaking.

While we have shown and described the embodiment in accordance with the present invention, it should be clear to those skilled in the art that further embodiments may be made without departing from the scope of the present invention.

What is claimed is:

1. A sanitary pad comprising:
an elongate body having a front end, a rear end and two divergent and smooth sides connected between the front end and the rear end, an absorbing area defined in a first side of the body and an area of the front end being narrower than an area of the rear end, an elongate reinforcement absorbing area located at the first side and close to the front end, an adherent layer applied to a second side of the body and located opposite to the first side.
